(19) United States
(12) Patent Application Publication
(10) Pub. No.: US 2003/0114923 A1
(43) Pub. Date: Jun. 19, 2003

(54) Graft and method of making

(76) Inventors: Thomas M. Swanick, Nashua, NH (US); Joseph Ferraro, Londonderry, NH (US); Ibrahim Dagher, Methuen, MA (US); Paul Martakos, Pelham, NH (US); Theodore Karwoski, Hollis, NH (US); Steven A. Herweck, Nashua, NH (US)

Correspondence Address: LAHIVE & COCKFIELD, 28 STATE STREET, BOSTON, MA, 02109, US

(21) Appl. No.: 10/295,333

(22) Filed: Nov. 14, 2002

Related U.S. Application Data
(60) Provisional application No. 60/335,937, filed on Nov. 14, 2001.

(51) Int. Cl. A61F 2/06

(52) U.S. Cl. 623/1.35; 264/239

(57) ABSTRACT

A graft has a seamless flow dividing structure. A method of manufacturing the flow dividing graft structure includes providing a first section of graft material having at least one side, a first end, and a second end. An opening is drawn out through the at least one side. A second section of graft material is coupled with the opening. An angled section is formed along the first section of graft material. The angled section provides a seamless division of flow supplied from the second section to the first section and directs the flow to each of the first and second ends of the first graft material. The resulting graft structure includes a main graft section. A branch graft section is coupled with the main graft section at an angled divider section. The angled divider section is seamless and is suitable for dividing flow through the flow dividing graft structure.

### RELATED APPLICATION

This application claims priority to, and the benefit of, co-pending U.S. Provisional Application No. 60/335,937, filed Nov. 14, 2001, for all subject matter common to both applications. The disclosure of said provisional application is hereby incorporated by reference in its entirety.

### FIELD OF THE INVENTION

The present invention relates to grafts suitable for replacing blood vessels, and more particularly to a method of manufacturing grafts resulting in vascular grafts of different configurations having seamless junctions with one or more branching graft legs.

### BACKGROUND OF THE INVENTION

Vascular grafts are routinely used to replace damaged or diseased blood vessels to restore blood flow. There are numerous configurations of vascular grafts available, some of which have one or more branches. Axillobifemoral and large diameter bifurcated grafts are examples of vascular grafts having one or more branches. In the case of axillobifemoral grafts, a side branch graft is attached to a main trunk section of the graft. In the case of large diameter bifurcated vascular grafts, a bifurcated section attaches to a larger diameter main trunk section of the graft.

There are several known methods of manufacturing a branched vascular graft. One such method can be summarized as a suturing process, the result of which is depicted in FIG. 1A. The example graft is made by W. L. Gore & Associates, Inc. as model #SB2001. The vascular graft 10 has a main trunk 12 section. The vascular graft 10 further includes a first branch 14 and a second branch 16. The first branch 14 is sutured together with the main trunk 12 at a first intersection 18. The second branch 16 is sutured together with the main trunk 12 at a second intersection 20. The method for manufacturing the vascular graft 10 illustrated begins with the formation of the main trunk 12. Each of the first branch 14 and the second branch 16 are formed separate from the main trunk 12. The first branch 14 is then sutured on to the main trunk 12 at the first intersection 18 and the second branch 16 is sutured on to the main trunk 12 at the second intersection 20. The sutures at the first intersection 18 and the second intersection 20 create small perforations, which would leak fluid, such as blood, passing through the vascular graft 10 unless sealed. Therefore, a sealant/adhesive 22 is applied to the exterior portion of the vascular graft 10 to seal the sutures and provide necessary reinforcement to the vascular graft 10.

FIG. 1B shows an internal view of the first intersection 18 and the second intersection 20 of FIG. 1A. Looking along the length of the main trunk 12, the first intersection 18 is on the left side and the second intersection 20 is on the right side of the vascular graft 10. The conventional method of manufacture results in a divider 13 positioned between each of the intersections 18 and 20. The divider 13 directs the fluid flow into each of the branches 14 and 16.

A different known method of manufacturing a vascular graft is described in U.S. Pat. No. 6,203,735B1 to Edwin et al. (Edwin '735). The method of shaping three-dimensional products involves manipulating an expanded polytetrafluoroethylene tubular body into a desired three-dimensional formation. The method includes radially expanding a longitudinally expanded polytetrafluoroethylene (ePTFE) tube to form a radially expanded ePTFE (rePTFE) tube, engaging the rePTFE tube circumferentially about a shaping mandrel. The assembly is heated to a temperature below the crystalline melt point temperature, or sintering temperature, of polytetrafluoroethylene to radially shrink the diameter of the rePTFE tube into intimate contact with the shaping mandrel. The assembly is then heated to a temperature above the crystalline melt point temperature of polytetrafluoroethylene to amorphously lock the microstructure of the shaped polytetrafluoroethylene body.

FIG. 1C depicts yet another conventional configuration for a textile or fabric graft 24. The graft 24 is made of a textile or fabric that is woven into the main trunk section 25 and legs 26 and 28. The weaving process leaves a hole at the point of the divider 27, which must be sewn together to seal the graft 24 and prevent leakage. This is often referred to as a seamless graft, but there is a small seam at the divider 27 location that must be sewn to prevent fluid leakage.

### SUMMARY OF THE INVENTION

There is a need for a seamless flow dividing graft structure and a corresponding method of making. The present invention is directed toward further solutions to address this need.

In accordance with one example embodiment of the present invention, a method of manufacturing a flow dividing graft structure includes providing a first section of graft material having at least one side, a first end, and a second end. An opening is drawn out through the at least one side. A second section of graft material is coupled with the opening. An angled section is formed along the first section of graft material. The angled section provides a seamless division of flow supplied from the second section to the first section and directs the flow to each of the first and second ends of the first graft material.

In accordance with further aspects of the present invention, the method includes expanding the first section of graft material prior to drawing out the opening. The method continues with placing the first section of graft material over a mandrel prior to drawing out the opening. The first section of graft material is restrained against the mandrel. The first section of graft material is shrink fit about the mandrel. Additional graft material is wrapped in a helix pattern about the first section of graft material and the mandrel. Additional graft material is then wrapped over the helix pattern and first section of graft material to form a second layer of graft material. The second layer of graft material is then restrained. The second layer of graft material, the helix, and the first section of graft material are heated. The opening drawn out through the at least one side includes drawing out a trunk, cutting a hole in the trunk, and removing the mandrel. The second graft section is installed on a second mandrel. First and second leg mandrels are installed onto the second mandrel. The first and second graft sections are heated. Additional graft material is wrapped around the second graft section. A cover of graft material is placed over the first and second graft sections. The cover of graft material is restrained. The cover of graft material is then shrink fit. The mandrel is removed to form the flow dividing graft structure.

In accordance with further aspects of the present invention, the flow dividing graft structure is formed of a hydrophobic, biocompatible, inelastic material. The flow dividing graft structure can also be formed of a bioresorbable material. The angled section can be sufficiently narrow to enable a reduced flow resistance and a reduced flow turbulence. The angled section can be monolithic. The flow dividing graft structure can be suitable to simulate anatomical physiological fluid flow divider conditions of a normal flow dividing hollow organ within a patient.

In accordance with another embodiment of the present invention, a method of manufacturing a flow dividing graft structure includes providing a section of graft material. The graft material is expanded, layered, and shrink fitted with additional graft material in a predetermined manner about a shape pattern to make one or more graft leg members seamlessly coupled with a main portion of the graft. The form is removed from the graft.

In accordance with another embodiment of the present invention, a flow dividing graft structure is provided. The structure includes a main graft section. A branch graft section is coupled with the main graft section at an angled divider section. The angled divider section is seamless and is suitable for dividing flow through the flow dividing graft structure.

In accordance with further aspects of the present invention, the flow dividing graft structure is formed of a hydrophobic, biocompatible, inelastic material. The flow dividing graft structure can also be formed of a bioresorbable material. The branch graft section can intersect with the main graft section to form the angled divider section which is sufficiently narrow to enable a reduced flow resistance and a reduced flow turbulence through the flow dividing graft structure. The angled divider section can be seamless. The angled divider section can be monolithic. The flow dividing graft structure can be suitable to simulate anatomical physiological fluid flow divider conditions of a normal flow dividing hollow organ within a patient.

In accordance with another embodiment of the present invention, a flow dividing graft structure formed by providing a section of graft material, expanding, layering, and shrink fitting the graft material with additional graft material in a predetermined manner about a shape pattern to make one or more graft leg members seamlessly coupled with a main portion of the flow dividing graft structure, and removing the shape pattern from the flow dividing graft structure is provided. The flow dividing graft structure includes a seamless monolithic structure having a main section. At least one seamlessly coupled branch section extends from the main section. The flow dividing graft structure includes a seamless flow divider junction between the main section and the at least one seamlessly coupled branch section.

In accordance with further aspects of the present invention, the flow dividing graft structure further includes a continuous monolithic junction and flow divider formed at each branch section having enhanced strength relative to conventional sewn seamed branch connection grafts.

### BRIEF DESCRIPTION OF THE DRAWINGS

The features and advantages of the present invention will become better understood with regard to the following description and accompanying drawings, wherein:

FIGS. 1A, 1B, and 1C are illustrations of vascular grafts produced according to conventional methods of manufacture;

FIG. 2 is an illustration of a vascular graft resulting from the process of one aspect of the present invention;

FIGS. 3A through 3C are a step-by-step illustration of a method of manufacture according to one aspect of the present invention;

FIG. 4 is an illustration of another vascular graft resulting from the process of another aspect of the present invention;

FIG. 5 is an illustration of an internal portion of a junction in accordance with the teachings of the present invention; and

FIG. 6 is a table comparing experimental results of graft performance.

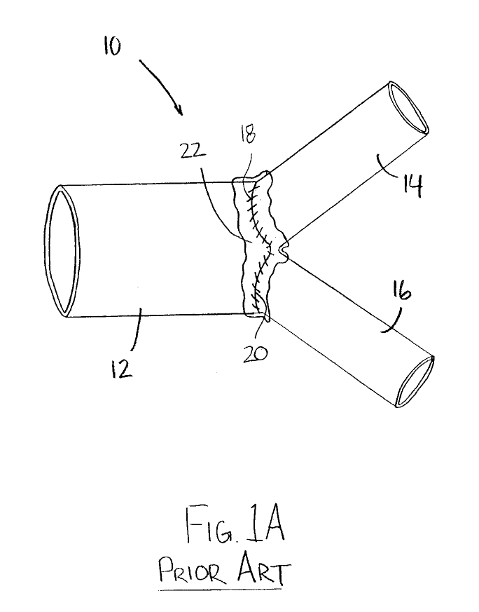

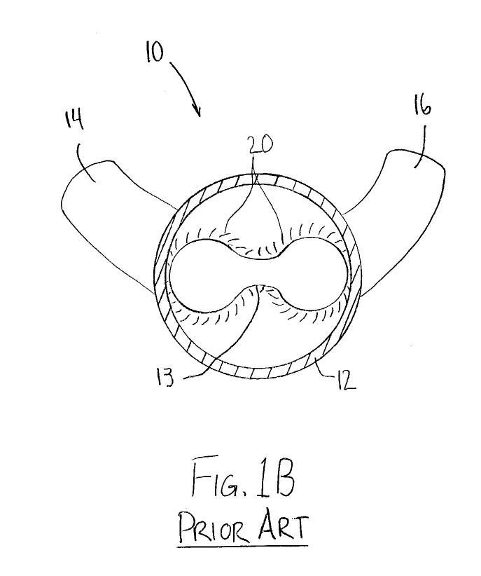

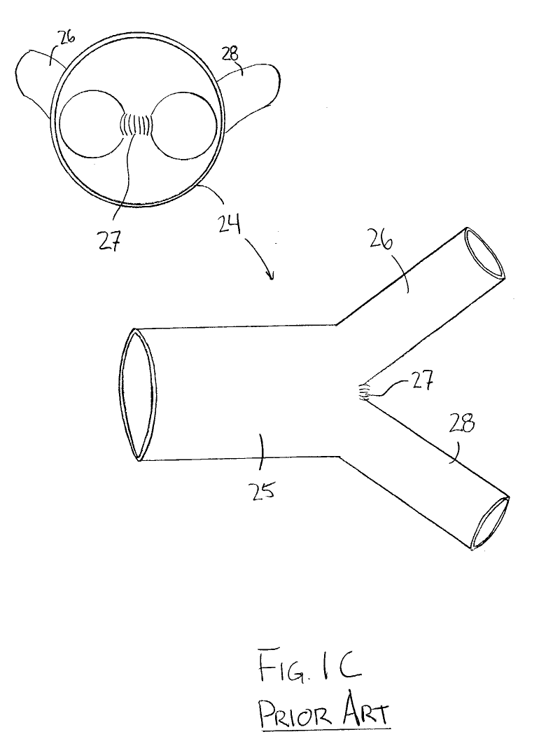

### DETAILED DESCRIPTION

An illustrative embodiment of the present invention relates to a vascular graft and corresponding method of making the vascular graft that is more efficient and results in a durable graft with seamless junctions. By “seamless” what is meant is a junction in which a seam is substantially imperceptible to fluid flowing through the graft, and does not contain holes or perforations from thread, sutures, or the like. The seamless junction differs from conventional grafts made of a fabric or textile that require a seamed connection between, for example, a main trunk section and a branch of a graft. Some conventional grafts address the holes of the seam with a reinforcement sewn over the seamed connection to cover the holes. Other conventional grafts may use a sealant on the exterior portions of the seam, preventing leakage through the holes formed by the seam, but leaving the seam and thread surface imperfections on the interior walls of the graft. None of the conventional solutions is seamless as intended by the teachings of the present invention.

The embodiments utilize a process to produce vascular grafts having one or more branches without the use of sutures for connecting the one or more branches. Sealants or adhesives are also not required to reinforce or seal the branch junctions. The inventive method results in seamless junctions, or angled sections, between a main trunk portion of the graft and one or more branches. The seamless junction in branched grafts represents a significant improvement in overall quality and integrity of the junction(s). The inventive method provides the ability to tailor junction shape and angle, which can result in improved flow at locations within the graft where branches re-direct flow through the graft. The improved flow dynamics at the branch junctions improve the long term clinical performance of the branched graft structure. In addition, the present method provides for the creation of an anatomically accurate junction, to better simulate and support normal, physiologic flow characteristics.

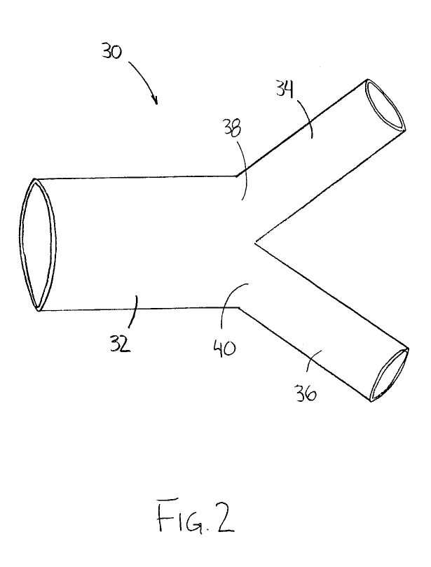

FIGS. 2 through 6, wherein like parts are designated by like reference numerals throughout, illustrate example embodiments of vascular grafts and a corresponding method of making according to the present invention, in addition to experimental test results. Although the present invention will be described with reference to the example embodiments illustrated in the figures, it should be understood that many alternative forms can embody the present invention. One of ordinary skill in the art will additionally appreciate different ways to alter the parameters of the embodiments disclosed, such as the size, shape, or type of elements or materials, in a manner still in keeping with the spirit and scope of the present invention.

FIG. 2 illustrates a graft 30 resulting from the method of manufacture according to the teachings of the present invention. The graft 30 includes a main trunk 32 section. The main trunk 32 branches out into a first leg 34 and a second leg 36, resulting in a bifurcated configuration. The main trunk 32 section represents a primary section, or starting point, from which other sections, such as legs 34 or 36, can extend. The main trunk 32 section does not need to be larger than the legs 34 and 36. Rather, the main trunk 32 section serves as a section that supports other sections. In the event a graft or structure made by the teachings of the present invention has a substantially symmetrical configuration with no clear primary section, the main trunk 32 section would be any one of the multiple sections making up the graft.

The first leg 34 branches off the main trunk 32 at a, angled section or first junction 38 and the second leg 36 branches off the main trunk 32 at another angled section or second junction 40. The first junction 38 and the second junction 40 are seamless transitions from the main trunk 32 section to each of the first leg 34 and the second leg 36, forming a monolithic structure. The term “monolithic” is meant to indicate that the resulting structure is formed of layers of material that are fused or bonded chemically or physically in a manner that prevents subsequent separation of the layers. The layers become a single structure that is effectively monolithic.

The main trunk 32 section maintains a larger diameter relative to the diameter of each of the first leg 34 and the second leg 36. The size and dimensions of the main trunk 32 section and each of the first leg 34 and the second leg 36 can vary depending on the application for the graft. Some uses may require larger diameter configurations, while other uses may require smaller diameter configurations. Likewise, the diameter and length of the first leg 34 can differ from the diameter and length of the second leg 36. The example graft 30 maintains dimensions of 18 mm×9 mm. One of ordinary skill in the art will appreciate that a graft with tapered dimensions can be constructed to better match patient anatomy or improve surgical technique. In addition, other dimensions for the graft 30 are possible, depending on a particular application.

The main trunk 32 section and the first leg 34 and second leg 36 are all formed of a biocompatible flexible material, such as, for example, expanded polytetrafluoroethylene (ePTFE). The ePTFE material is a hydrophobic, biocompatible, inelastic material having a low coefficient of friction. Alternatively, the biocompatible material can be constructed from a bioresorbable material, such as polyglycolic acid polymers, polycaprolactone polymers, polylactic acid polymers, or copolymer combinations thereof. Any material can be used to form a vascular graft that is suitable as a substitution for vessels that carry or circulate fluids within a body, and is compatible with the process of the present invention for manufacture of the graft with seamless junctions.

The method of the present invention can also form other types of grafts, such as axillofemoral, axillobifemoral, coronary arterial, bifurcated, and trifurcated configurations. The ePTFE can serve as the material to form these other types of grafts, in addition to other suitable materials, depending on the application of the graft.

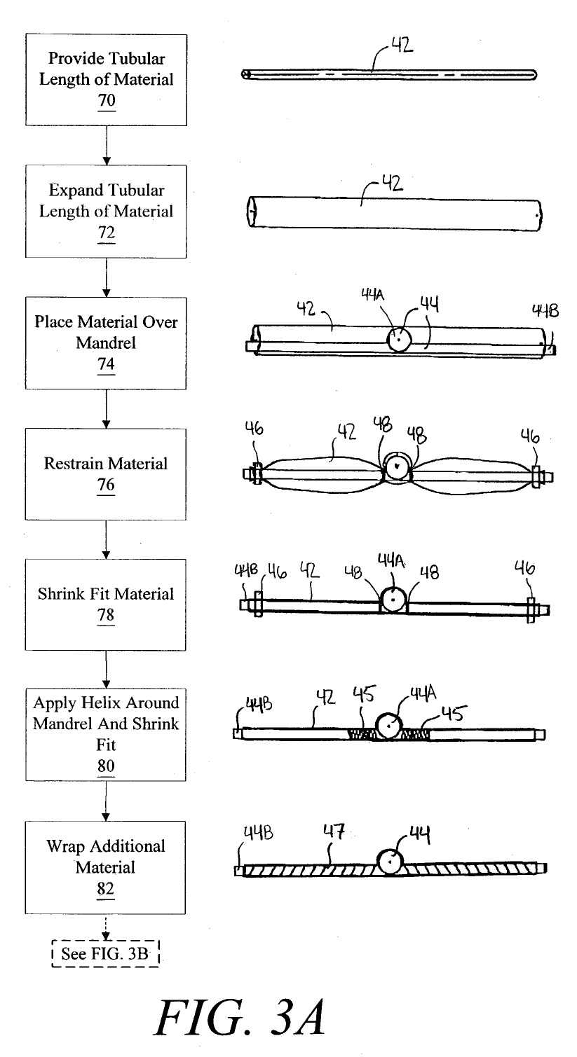

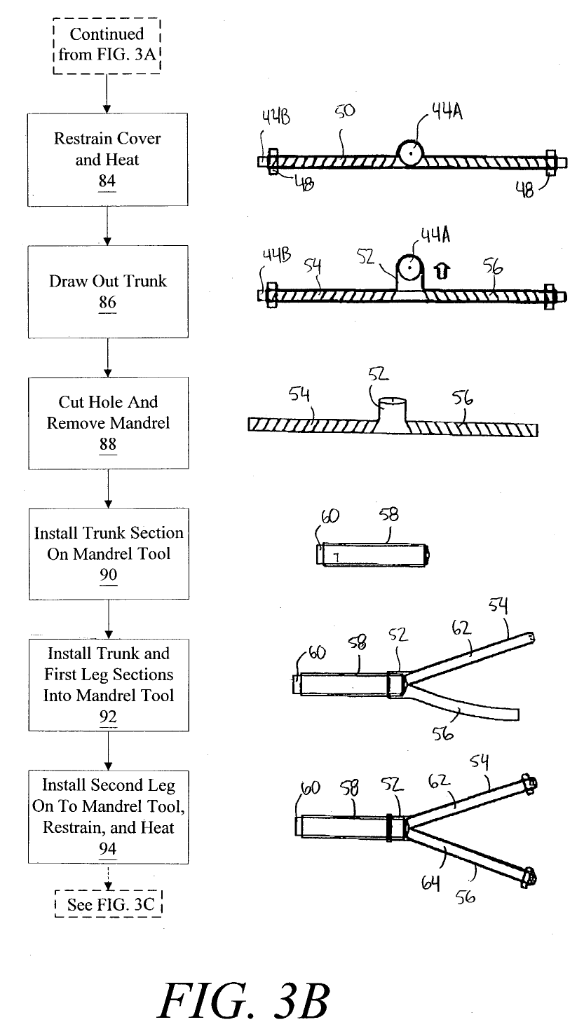

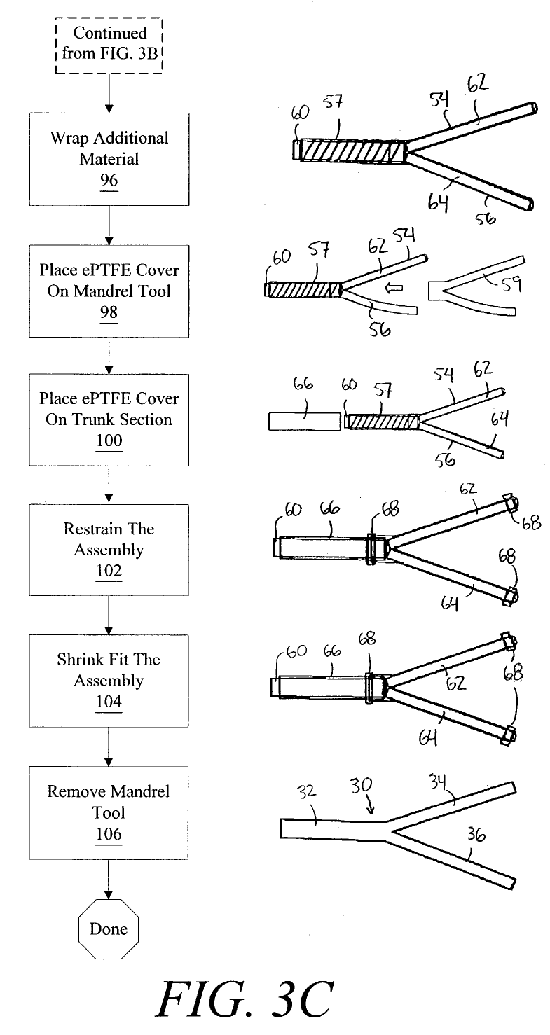

FIGS. 3A through 3C show a stepwise illustration of a method for manufacturing the graft 30 of FIG. 2, in addition to grafts of other configurations. The example illustrated herein forms the graft from ePTFE material, but other suitable materials can be utilized as understood by one of ordinary skill in the art. In addition, the method of the present invention can be executed by hand, by machine, or by combination of both hand and machine.

The method begins with providing a length 42 of tubular ePTFE material at a diameter about equal to a desired diameter for the smallest of the legs being formed by the method (step 70). The length 42 of tubular ePTFE is expanded to a diameter approximately equal to a desired diameter for the main trunk 32 section (step 72). The expanded length 42 of tubular ePTFE is then placed over a two-piece mandrel 44 (step 74) which contains a ball insert 44A and a bar insert 44B. Restraining mechanisms 46 bind the ends of the expanded length 42, and additional restraining mechanisms 48 bind portions of the expanded length 42 around a central portion of the mandrel 44 (step 76). A shrink fitting process shrinks the expanded length 42 onto the mandrel 44 with applied heat (step 78). The heat applied for ePTFE material can be in the range between 330 and 380 degrees Celsius, for a period of about four to ten minutes.

The method continues with the removal of the restraining mechanisms 46 and 48 and the wrapping of additional ePTFE material 45 in a helix fashion about the length 42 on either side of the mandrel 44 and heat fused to the graft by a heat treatment in which heat is applied to the assembly in the range of 330 to 380 degrees Centigrade for a period of about four to ten minutes (step 80). A wrapping of additional ePTFE material 47 is then applied across the length 42 (step 82). The wrapped additional material 47 is restrained as in step 76, and heat is applied in the range between 330 and 380 degrees Centigrade, for a period of about ten to twenty minutes. The heat causes the wrapped additional material 47 to shrink fit around the assembly (step 84).

The additional wrap material utilized in the method of the present invention can be formed of a hydrophobic, biocompatible, inelastic material, such as ePTFE. Alternatively, the wrap material can be constructed from a bioresorbable material, such as polyglycolic acid polymers, polycaprolactone polymers, polylactic acid polymers, or copolymer combinations thereof.

The restraining mechanisms 48 are removed and a trunk profile is created by drawing or pulling the ball insert 44A out and away from the bar insert 44B of the two piece mandrel 44 to create a trunk 52 profile, a first leg 54, and a second leg 56 (step 86). A hole is cut in the trunk 52 profile and the ball insert 44A is removed, followed by the removal of the bar insert 44B through the hole in the first leg 54 or second leg 56 (step 88).

A trunk section 58 is installed on to a bifurcate mandrel trunk tool 60 (step 90). The first leg 54 and trunk 52 are installed on to the bifurcate mandrel trunk tool 60 and a first bifurcate mandrel leg tool 62 (step 92). A second bifurcate mandrel leg tool 64 then slides through the second leg 56 and couples with the bifurcate mandrel trunk tool 60, and the assembly is restrained and heat treated between 330 and 380 degrees Centigrade for a period of about ten to twenty minutes (step 94). A wrap 57 is installed around the bifurcate mandrel trunk tool 60 (step 96). The second bifurcate mandrel leg tool 64 is then removed and an ePTFE cover material 59, prepared as in steps 86 and 88, is placed on to the mandrel 44 (step 98). The second bifurcate mandrel leg tool 64 is re-installed and an ePTFE cover 66 is installed over the trunk section 58 (step 100). The entire assembly is restrained using restraining mechanisms 68 (step 102). The entire assembly is then shrink fit onto the bifurcate mandrel trunk tool 60 and leg tools 62 and 64 (step 104). The heat applied to the assembly ranges between 330 and 380 degrees Centigrade, for a period of about fifteen to thirty minutes.

The first bifurcate mandrel leg tool 62 and the second bifurcate mandrel leg tool 64 are removed from the bifurcate mandrel trunk tool 60 and the first leg 54 and second leg 56. The bifurcate mandrel trunk tool 60 is then removed (step 106). The desired bifurcated graft 30 remains.

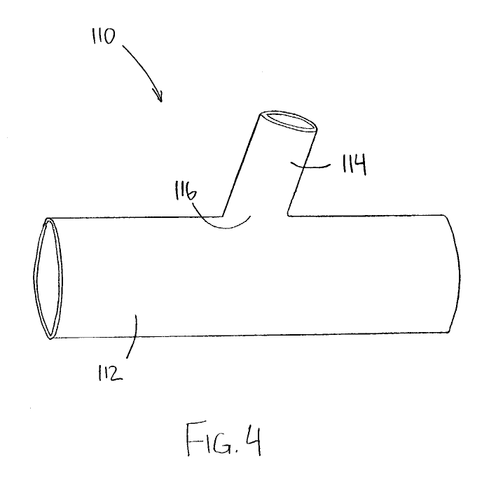

One of ordinary skill in the art will appreciate that the teachings of the present invention can result in the formation of grafts of a number of different configurations. For example, FIG. 4 illustrates a graft 110 having a single branch or leg 114 extending from a main trunk 112. The graft 110 is made in accordance with the method of the present invention, thus there is a seamless junction 116 connecting the leg 114 with the main trunk 112. The number, shape, size, location, and dimension of legs branching off the main trunk portion can vary as understood by one of ordinary skill in the art. The teachings of the present invention enable the design of a monolithic graft having seamless junctions and having one or more sections of predetermined dimensions as desired.

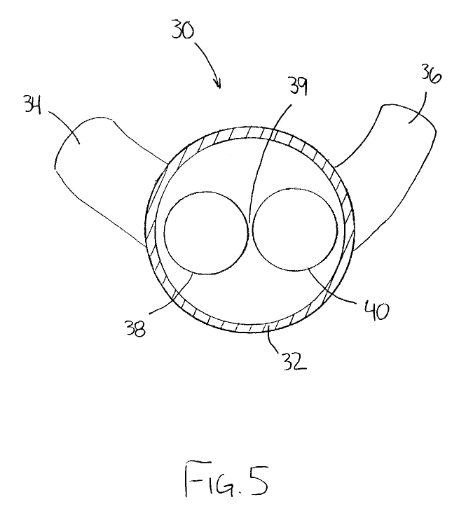

FIG. 5 illustrates an internal view of the first junction 38 and the second junction 40 of FIG. 2. The view looks into the larger end of the main trunk 32. Looking along the length of the trunk 32, the first junction 38 is on the left side and the second junction 40 is on the right side of the graft 30. The first junction 38 leads to the first leg 34, and the second junction leads to the second leg 36. The method of the present invention enables a divider 39 between each of the junctions 38 and 40 and the legs 34 and 36 to be narrow relative to other conventional grafts. The narrow characteristic of the divider 39 allows for a more efficient control of fluid flow through the graft 30, and substantially reduces resistance to fluid flow and associated turbulence. The narrow divider 39 thus enables a relatively smoother flow at the transition from the trunk 32 to the legs 34 and 36. The narrow divider 39 further provides for a more physiologically accurate flow characteristics through the graft 30.

The narrow divider 39 made in accordance with the teachings of the present invention is a seamless divider 39. There are no perforations or threads from sutures. The divider 39 is a seamless and monolithic feature that can efficiently and effectively divide and distribute a fluid flowing past the divider 39. The absence of a seam enhances the strength of the divider 39 and results in a more durable graft that is able to withstand relatively higher fluid pressures relative to conventional grafts.

The inventive method of the present invention utilizes a process to produce products having one or more branches or legs without the use of sutures. The method thus results in a monolithic structure without seams. The size, shape, and the angle of the branches or legs can vary, and can be tailored for specific applications.

The seamless monolithic structure also promotes improved flow dynamics. Anatomically correct flow characteristics can be reproduced in a graft made in accordance with the teachings of the present invention.

Other know bifurcated grafts have developed kinks at the legs due to repetitive longitudinal force exerted on the legs by the beating aorta, and by external compression forces exerted by internal organs. The structure of the present invention significantly reduces graft kinking and abrasion of surrounding internal organs when implanted. The ePTFE is formed of a microstructure of nodes and fibrils that provide radial support integral to the graft wall. The microstructure provides the enhanced kink resistance and minimizes organ abrasion.

Grafts made in accordance with the teachings of the present invention offer enhanced junction strength as well. For example, on a 16 mm×8 mm graft, junction strength can approach about 54 lbs. of pressure. This is a significant increase over other known graft devices, some of which are limited by the strength of sutures used to create the intersection or junction, in combination with adhesive or sealant.

The teachings of the present invention provide for the enhanced junction strength in that the main trunk section and leg sections are formed such that the coupling of these sections occurs at locations other than major areas of stress concentration during use. In other words, one major area of stress caused by fluid flow is the divider 39. In other conventional grafts, the divider includes perforations and threads from sutures which weaken the overall strength of the graft. The present invention makes use of a seamless junction and seamless divider 39 that enhance the strength of the graft because they contain no perforations.

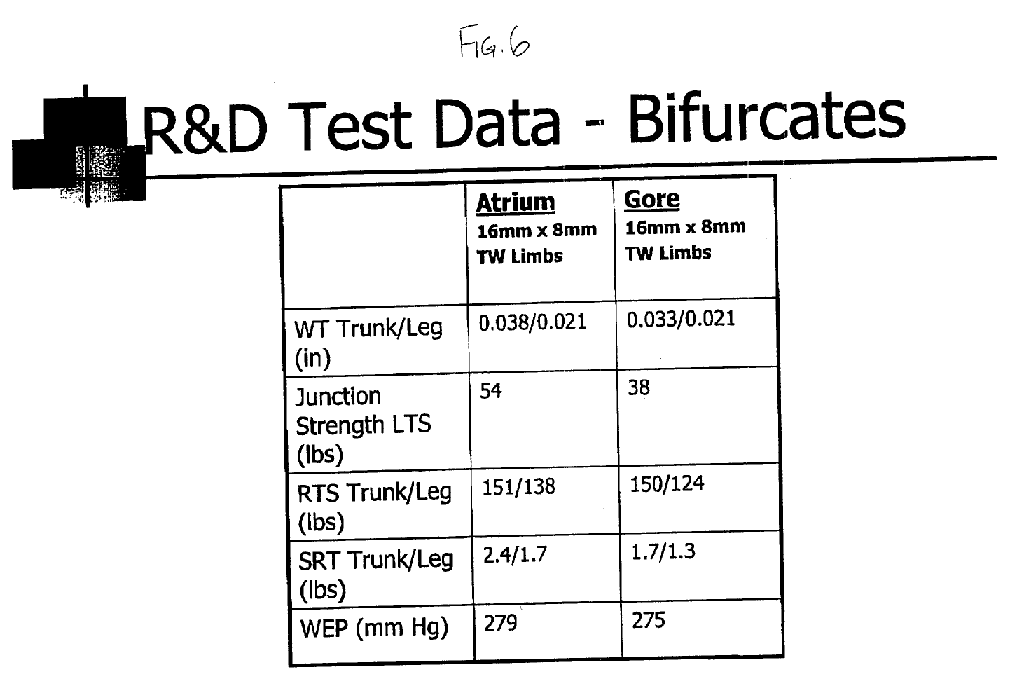

The increased junction strength is evidenced by trial experiments performed by Atrium Corporation of Hudson, N.H. and displayed in the table of FIG. 6. The table illustrates results obtain from tests performed on a prototype Atrium graft (Atrium graft) made in accordance with the teachings of the present invention and a sample graft made by W. L. Gore & Associates, Inc. having model number SB2001 (Gore graft). Both grafts were 16 mm×8 mm thin wall grafts. The wall thickness (WT) in the trunk and leg portions was as indicted in the table. A tensile force was applied to each graft using a commercially available tensile test apparatus made by Instron Corp., which measures force to yield the material to failure. Evidence of material or junction failure was observed at different force values. The Atrium graft was able to withstand 54 lbs. of pressure at each junction, representing longitudinal tensile strength (LTS), while the Gore graft withstood 38 lbs. of pressure. The Atrium graft had a radial tensile strength (RTS) of 151 lbs. in the trunk and 138 lbs. in the leg, while the Gore graft had an RTS of 150 lbs. in the trunk and 124 lbs. in the leg. The Atrium graft had a suture retention strength (SRT) of 2.4 lbs. in the trunk and 1.7 lbs. in the leg, while the Gore graft had an SRT of 1.7 lbs. in the trunk and 1.3 lbs. in the leg. The water entry pressure (WEP) withstood by the Atrium graft was 279 mm Hg, while the WEP withstood by the Gore graft was 275 mm Hg. The experimental data suggests that the Atrium graft has a relatively greater strength in all areas measured relative to the sample Gore graft.

Numerous modifications and alternative embodiments of the present invention will be apparent to those skilled in the art in view of the foregoing description. Accordingly, this description is to be construed as illustrative only and is for the purpose of teaching those skilled in the art the best mode for carrying out the present invention. Details of the structure may vary substantially without departing from the spirit of the invention, and exclusive use of all modifications that come within the scope of the appended claims is reserved.

What is claimed is:

1. A method of manufacturing a flow dividing graft structure, comprising:
providing a first section of graft material having at least one side, a first end, and a second end;
drawing out an opening through the at least one side;
coupling a second section of graft material with the opening; and
forming an angled section along the first section of graft material;
wherein the angled section provides a seamless division of flow supplied from the second section to the first section and directs the flow to each of the first and second ends of the first graft material.

2. The method of claim 1, further comprising expanding the first section of graft material prior to drawing out the opening.

3. The method of claim 1, further comprising placing the first section of graft material over a mandrel prior to drawing out the opening.

4. The method of claim 3, further comprising restraining the first section of graft material against the mandrel.

5. The method of claim 4, further comprising shrink fitting the first section of graft material.

6. The method of claim 5, further comprising wrapping additional graft material in a helix pattern about the first section of graft material and the mandrel.

7. The method of claim 6, further comprising wrapping additional graft material over the helix pattern and first section of graft material to form a second layer of graft material.

8. The method according to claim 7, further comprising restraining the second layer of graft material.

9. The method according to claim 8, further comprising heating the second layer of graft material, the helix, and the first section of graft material.

10. The method according to claim 9, wherein drawing out the opening through the at least one side comprises drawing out a trunk, cutting a hole in the trunk, and removing the mandrel.

11. The method according to claim 10, further comprising installing the second graft section on a second mandrel.

12. The method according to claim 11, further comprising installing first and second leg mandrels onto the second mandrel.

13. The method according to claim 12, further comprising heating the first and second graft sections.

14. The method according to claim 13, further comprising wrapping additional graft material around the second graft section.

15. The method according to claim 14, further comprising placing a cover of graft material over the first and second graft sections.

16. The method according to claim 15, further comprising restraining the cover of graft material.

17. The method according to claim 16, further comprising shrink fitting the cover of graft material.

18. The method according to claim 17, further comprising removing the mandrel to form the flow dividing graft structure.

19. The method according to claim 1, wherein the flow dividing graft structure is formed of a hydrophobic, biocompatible, inelastic material.

20. The method according to claim 1, wherein the flow dividing graft structure is formed of a bioresorbable material.

21. The method according to claim 1, wherein the angled section is sufficiently narrow to enable a reduced flow resistance and a reduced flow turbulence.

22. The method according to claim 1, wherein the angled section is monolithic.

23. The method according to claim 1, wherein the flow dividing graft structure is suitable to simulate anatomical physiological fluid flow divider conditions of a normal flow dividing hollow organ within a patient.

24. A method of manufacturing a flow dividing graft structure, comprising:
providing a section of graft material;
expanding, layering, and shrink fitting the graft material with additional graft material in a predetermined manner about a shape pattern to make one or more graft leg members seamlessly coupled with a main portion of the graft; and
removing the form from the graft.

25. The method according to claim 24, wherein the flow dividing graft structure is formed of a hydrophobic, biocompatible, inelastic material.

26. The method according to claim 24, wherein the flow dividing graft structure is formed of a bioresorbable material.

27. The method according to claim 24, wherein the one or more graft leg members intersects with the main portion of the graft to form an angled section sufficiently narrow to enable a reduced flow resistance and a reduced flow turbulence.

28. The method according to claim 27, wherein the angled section is seamless.

29. The method according to claim 27, wherein the angled section is monolithic.

30. The method according to claim 24, wherein the flow dividing graft structure is suitable to simulate anatomical physiological fluid flow divider conditions of a normal flow dividing hollow organ within a patient.

31. A flow dividing graft structure, comprising:
a main graft section; and
a branch graft section coupled with the main graft section at an angled divider section;
wherein the angled divider section is seamless and is suitable for dividing flow through the flow dividing graft structure.

32. The flow dividing graft structure of claim 31, wherein the flow dividing graft structure is formed of a hydrophobic, biocompatible, inelastic material.

33. The flow dividing graft structure of claim 31, wherein the flow dividing graft structure is formed of a bioresorbable material.

34. The flow dividing graft structure of claim 31, wherein the branch graft section intersects with the main graft section to form the angled divider section which is sufficiently narrow to enable a reduced flow resistance and a reduced flow turbulence through the flow dividing graft structure.

35. The flow dividing graft structure of claim 31, wherein the angled divider section is seamless.

36. The method according to claim 31, wherein the angled divider section is monolithic.

37. The method according to claim 31, wherein the flow dividing graft structure is suitable to simulate anatomical physiological fluid flow divider conditions of a normal flow dividing hollow organ within a patient.

38. A flow dividing graft structure formed by providing a section of graft material, expanding, layering, and shrink fitting the graft material with additional graft material in a predetermined manner about a shape pattern to make one or more graft leg members seamlessly coupled with a main portion of the flow dividing graft structure, and removing the shape pattern from the flow dividing graft structure, the flow dividing graft structure comprising:
a seamless monolithic structure having a main section; and
at least one seamlessly coupled branch section extending from the main section;
wherein the flow dividing graft structure includes a seamless flow divider junction between the main section and the at least one seamlessly coupled branch section.

39. The flow dividing graft structure in accordance with claim 38, further comprising a continuous monolithic junction and flow divider formed at each branch section having enhanced strength relative to conventional sewn seamed branch connection grafts.

40. The flow dividing graft structure in accordance with claim 38, wherein the flow dividing graft structure is suitable to simulate anatomical physiological fluid flow divider conditions of a normal flow dividing hollow organ within a patient.
